(19) United States
(12) Patent Application Publication
(10) Pub. No.: US 2013/0204037 A1
(43) Pub. Date: Aug. 8, 2013

(54) Catalytic alkylation of alcohols to liquid ethers and organic compounds to alkylated products

(71) Applicant: Melvin Keith Carter, Lincoln, CA (US)

(72) Inventor: Melvin Keith Carter, Lincoln, CA (US)

(73) Assignee: Carter Technologies, Lincoln, CA (US)

(21) Appl. No.: 13/385,192

(22) Filed: Feb. 7, 2012

(51) Int. Cl. C07C 41/09; C07C 45/68; C07C 51/367; C07C 209/02

(52) U.S. Cl. 562/473; 568/698; 564/480; 568/658; 564/479; 568/388

(57) ABSTRACT

A catalytic process is taught for non-oxidative alkylation of organic compounds, comprising alcohols, alkanes, glycols, ethers, aldehydes, ketones, carboxylic acids, esters, amines, thiols or phosphines, by alkyl groups produced from alcohols or glycols, forming products comprising ethers and other higher molecular weight alkylated compounds. The process is conducted at a reflux temperature below 200° C. in the presence of an acid, alkali or neutral salt dehydrating agent comprising sulfuric acid, phosphoric acid or their salts, lime or anhydrous calcium sulfate in the absence of zero valent metals and air. Specifically, this catalytic process converts ethanol to ethyl butyl ethers, ethyl hexyl ethers and dibutyl ethers or oxygenated gasoline as well as amines comprising n-butyl amine plus butanol to dibutyl amine and butyl hexyl amines at ambient pressure. This same catalytic alkylation chemistry, which does not constitute a condensation reaction, alkylates 4-hydroxybenzoic acid using ethanol to 4-ethoxyethylbenzoic acid products.

### BACKGROUND

1. Field of Invention

This invention teaches ambient pressure catalytic alkylation of organic compounds, comprising alcohols, alkanes, glycols, ethers, aldehydes, ketones, carboxylic acids, esters, amines, thiols or phosphines, by alkyl groups produced from alcohols or glycols forming products comprising ethers and other higher molecular weight alkylated compounds at a reflux temperature below 200° C. in the presence of a minor amount of an acid, alkali or neutral salt dehydrating agent comprising sulfuric acid, phosphoric acid or their salts, or respectively lime or anhydrous calcium sulfate in the absence of zero valent metals and air. Specifically, this application discloses efficient catalytic alkylation of chemical compounds produced from renewable resources including liquid ethanol and/or fusil oils that include higher molecular weight alcohols to ethers or oxygenated gasoline at reflux temperatures employing catalysts based on selected mono-metal, di-metal, tri-metal and/or poly-metal backbone or molecular string type transition metal complexes of low valence possessing a degree of symmetry as described herein.

2. Description of Prior Art

The chemical process industry has grown to maturity based on petroleum feed stocks. Petroleum is a non-renewable resource that may become unavailable in the next 100 years. This planet Earth fosters continual growth of numerous carbohydrate based plants including fruits, vegetables and grain food sources plus their supporting plant stalks, trees and related natural waste materials for recycle. Grains, corn cobs, wood, support plant stalks and certain grasses are, in part, subject to direct catalytic chemical conversion and/or bio-fermentation processes producing ethanol and related products. A major industry is blooming in ethanol production by conversion of corn and grain materials where much of the product is sold as combustion engine fuel or its additive. Ethanol is becoming more available as a renewable resource and this application teaches its catalytic conversion to valuable oxygenated gasoline and chemical intermediates for use in the automotive fuels and chemical process industries.

A number of chemical reaction paths have previously been taught for conversion of aliphatic alcohols to higher molecular weight alcohols and related products using carbonyl insertion, partial oxidation and other gas phase processes but do not teach high conversion efficiencies to ethers in liquid form. Gaseous ethanol has been converted to diethyl ether at 120° C. and to ethylene gas at 180° C. over a dehydrating acid in the absence of a transition metal catalyst. Controlled oxidation of methane at high temperatures, previously investigated under a wide range of conditions, has produced carbon dioxide, carbon monoxide, low concentrations of unsaturated hydrocarbons, oligomers, low levels of alcohols, aldehydes and water. However, these efforts have not produced significant amounts of aldehydes, alcohols or liquid ether products. As a result direct conversion of saturated hydrocarbons to aldehydes and/or alcohols has essentially been abandoned in favor of conversion of more labile hydrocarbons such as alkenes or vinyl alcohols that possess more reactive groups. Secondary and tertiary alcohols can be produced from branched olefins in the gas phase by combining with water or primary alcohols in the presence of sulfuric acid. Higher boiling branched alcohols have been produced from primary alcohols over sodium alkoxide in the presence of a nickel catalyst at 200° C. to 250° C. in the gas phase by means of the Guerbet reaction. Alcohols can also be dehydrated to form olefins over aluminum oxide at 350° C. to 450° C. in the gas phase at elevated pressure.

There are several other hot tube reactions described in the scientific and patent literature for conversion of gaseous alcohols to a wide range of low concentration products from gasoline type hydrocarbons to aldehydes and ethers. Aldehydes and ketones can be formed by passing alcohol vapors over Cu and its alloys or Ag at 300° C. to 600° C. in the presence of controlled amounts of air. U.S. Pat. No. 6,166,265, issued Dec. 26, 2000, introduced a process for preparation of n-butyraldehyde and/or n-butanol by reacting butadiene with vaporized alcohol at super-atmospheric pressure and elevated temperatures using an acid resin or one of several transition metal oxides. U.S. Pat. No. 6,350,918, issued Feb. 26, 2002, teaches a process for the selective oxidation of alcohols to aldehydes in the vapor phase at 150° C. to 600° C. over oxides of V, Cr, Mo, W or Re in their high oxidation states well above 2+. Less selective chemistry may oxidize vaporized alcohol to aldehydes and ketones. Aldehydes can also be produced by a chemical exchange where one oxidized organic compound may transfer its oxygen atoms to an alcohol converting it to an aldehyde.

Alkyl ethers are commonly produced from branched hydrocarbons in a distillation process at elevated temperature and pressure. Alkyl tertiary butyl ethers have been produced in this manner for application as gasoline additives. U.S. Pat. No. 6,107,526, issued Aug. 22, 2000, disclosed addition of ethanol to iso-butene (from dehydration of iso-butane) in contact with a catalyst at 65 to 185 pounds per square inch pressure and 30° C. to 75° C. in a gas phase in formation of ethyl tertiary butyl ether during a distillation process.

Catalytic chemistries have also been taught in the production of other products. Vapor phase alkylation of aromatic amines over an iron oxide/titanium oxide catalyst performed at 300° C. to 400° C. was reported in U.S. Pat. No. 5,986,138, issued Nov. 16, 1999. U.S. Pat. No. 6,348,619, issued Feb. 19, 2002, disclosed the formation of esters wherein oxygen or air is passed through an alcohol plus a selected aldehyde at 0° C. to 100° C. at elevated pressure over a palladium-bismuth-lead catalyst.

The above reported chemistries have been conducted primarily in the gas phase at high pressure in an oxidative environment and are, therefore, distinctly different from catalytic alkylations conducted in the liquid phase at ambient pressure in a non-oxidative environment as taught herein. The invention disclosed in this application teaches non-oxidative catalytic alkylation of liquid alcohols, glycols, amines and phenols to related alkylated organic products including alkyl ethers or oxygenated gasoline using mono-metal, di-metal, tri-metal and/or poly-metal backbone or molecular string type transition metal catalysts in a low valance state. In addition, amines, diamines and polyamines may be alkylated and/or converted to higher molecular weight amines. It also discloses non-oxidative catalytic conversion of 4-hydroxybenzoic acid dissolved in ethanol to 4-ethoxybenzoic acid compounds by means of alkylation chemistry. No labile or other reactive chemical groups are required, although dehydrating agents were employed. Use of selected mono-metal, di-metal, tri-metal and/or poly-metal backbone or molecular string type transition metal catalysts produced in the absence of air, described in this application, result in high yields of the reported products.

### SUMMARY OF THE INVENTION

This invention describes non-oxidative catalytic chemical alkylation methods using selected members of transition metal catalysts in their low valence states, possessing a degree of symmetry, for alkylation of liquid alcohols to liquid ethers or oxygenated gasoline products, comprising ethanol to ethyl butyl ethers, ethanol plus ethyl butyl ethers to ethyl hexyl ethers and dibutyl ethers as oxygenated gasoline, ethanol plus ethyl hexyl ethers to butyl hexyl ethers as oxygenated gasoline, n-butyl amine plus butanol to dibutyl amine, butyl hexyl amines and related amines at ambient pressure. This same catalytic chemistry also converts 4-hydroxybenzoic acid plus ethanol as the alkylating agent to 4-ethoxybenzoic acid. This catalytic chemical conversion process operates throughout the liquidus range of the reactants.

It is an object of this invention, therefore, to provide a non-oxidative mono-metal or molecular string transition metal catalytic process facilitating conversion of alcohols to ether products. It is another object of this invention to catalytically alkylate organic compounds to higher molecular weight products. It is still another object of this invention to catalytically alkylate amines to higher molecular weight amine products at ambient pressure. It is still another object of this invention to catalytically convert liquid alcohols to oxygenated gasoline at reflux temperatures. Other objects of this invention will be apparent from the detailed description thereof which follows, and from the claims.

### DETAILED DESCRIPTION OF THE INVENTION

A process for non-oxidative chemical alkylation of liquid compounds comprising ethanol to ethyl butyl ethers, ethanol plus ethyl butyl ethers to dibutyl ether and ethyl hexyl ethers as oxygenated gasoline, ethanol plus ethyl hexyl ethers to butyl hexyl ethers as oxygenated gasoline, n-butyl amine plus butanol to dibutyl amine, butyl octyl amines and related products at ambient pressure. This same catalytic chemistry also converts substituted liquid organic compounds comprising aldehydes, ketones, ethers, phenols and amines to higher molecular weight alkylated organic products in the absence of air. This process employs transition metal compounds, comprising [vanadium(II)]₂, [chromium(II)]₂, [manganese(II)]₂, [cobalt(II)]₂ and catalysts based on other transition metals type compounds, for which the transition metals and directly attached atoms possess C₄ᵥ, D₄ₕ or D_{2d} point group molecular symmetry. These catalysts have been designed based on a formal theory of catalysis, and the catalysts have been produced, and tested to prove their activity. The theory of catalysis rests upon a requirement that a catalyst possess a single metal atom or a molecular string such that transitions from one molecular electronic configuration to another be barrier free so reactants may proceed freely to products as driven by thermodynamic considerations. Catalysts effective for chemical conversion of liquid alcohols to liquid ethers can be made from mono-metal, di-metal, tri-metal and/or poly-metal backbone or molecular string type compounds of the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof. These catalysts are made in the absence of oxygen so as to produce compounds wherein the oxidation state of the transition metal is low, typically monovalent or divalent. Anions employed for these catalysts comprise fluoride, chloride, bromide, iodide, cyanide, isocyanate, thiocyanate, sulfate, phosphate, oxide, hydroxide, oxalate, acetate and organic chelating agents. Mixed transition metal compounds were also effective catalysts for non-oxidative chemical conversions.

These catalysts act on alcohols, alkanes, glycols, aldehydes, ketones, ethers, amines and phenol compounds to generate free radicals in times believed to be the order of or less than that of a normal molecular vibration. This may be viewed in a classical sense as generation of free radicals in equilibrium as indicated hereinafter, namely CH₃CH₂OH⇄CH₃CH₂.+.OH, CH₃CH₂OH⇄CH₃CH₂O.+.H radicals for alkylation of and alkyl ether addition to available organic reactants. Catalytic exposure causes ethanol, propanol, butanol and ethylene glycol to become alkylating agents provided the water by product is removed from the reaction sphere. Thus, ethanol, the exemplary compound applied throughout this application, reacts with itself to produce ethyl butyl ether, dibutyl ethers, ethyl hexyl ethers and higher molecular weight ether compounds plus water wherein catalytically generated alkyl radicals attack both ethyl and hydroxyl sites, and water so formed is eliminated in the presence of a minor amount of an acid, alkali or neutral salt dehydrating agent comprising sulfuric acid, phosphoric acid or their salts, or respectively lime or anhydrous calcium sulfate in the absence of zero valent metals and air. This catalytic reaction chemistry is an alkylation for if it were a condensation then ethanol would have produced diethyl ether rather than ethyl butyl ether, dibutyl ethers, ethyl hexyl ethers and higher molecular weight ether compounds.

Ethanol mixed in roughly equal molar concentrations with other compounds alkylates them, specifically ethoxylates them, and produces a wide range of products. Ethanol reacts with itself or normal butanol in the presence of a selected catalyst to produce ethyl butyl ether, ethyl hexyl ether, dibutyl ethers and oxygenated gasoline products plus water. Ethanol reacts with a ketone such as acetone to form 2-pentanone and an allyl ether plus water. Ethanol plus normal butyl amine produces ethyl butyl amine, ethyl hexyl amines and dibutyl amines plus water. Ethanol plus normal butyl thiol produces ethyl butyl sulfide, ethyl hexyl sulfides and dibutyl sulfides plus water. Ethanol plus normal butyl phosphine produces ethyl butyl phosphines, ethyl hexyl phosphines, and dibutyl phosphines plus water.

### Catalyst Selection Considerations

The Concepts of Catalysis effort formed a basis for selecting molecular catalysts for specified chemical reactions through computational methods by means of the following six process steps. An acceptable chemical conversion mechanism, involving a single or pair of transition metal atoms, was established for the reactants (step 1). A specific transition metal, such as cobalt, was selected as a possible catalytic site as found in an M or M-M string (step 2), bonded with reactant molecules in a C₄ᵥ, D_{2d} or D₄ₕ point group molecular symmetry configuration, and having a computed bonding energy to the associated reactants of −5 to −60 kcal/mol (step 3). The first valence state for which the energy values were two-fold degenerate was 2+ in most cases although 1+ and 3+ have been produced (step 4). Cyanide, chloride and other anions may be chosen provided they are chemically compatible with the metal in formation of the catalyst (step 5). An inspection should also be conducted to establish compliance with the rule of 18 (or 32) to stabilize the catalyst; thus, compatible ligands may be added to complete the coordination shell (step 6). This same process may be applied for selection of a catalyst using any of the first, second or third row transition metals, however, only those with acceptable negative bonding energies can produce effective catalysts. The approximate relative bonding energy values have been computed using a semi-empirical algorithm. This computational method indicated that any of the first row transition metal complexes produced usable catalysts once the outer coordination shell had been completed with ligands, even though only the elements Ti, V, Cr, Mn, Co, Ni and Cu indicated reasonable bonding energies for the first row transition metals in a simplified molecular model. In general, preliminary energy values computed for transition metal ethanol complexes produced useable catalysts once bonding ligands had been added.

Catalyst structures including a pair of bonded transition metal atoms require chelating ligands and/or bonding orbital structures that are different for each metal. The following compounds comprise a limited selection of examples. For the first row transition metals vanadium catalysts comprise vanadium(II) oxide, (VOSO₄)₂, and (VF₂)₂ having V—V bonds and ethylenediamine (EDA) links the metals in (VCl₂)₂EDA₂ while ethanol displaces a CO and/or a THF in the compound [V(THF)₄Cl₂][V(CO)₆]₂. Chromium catalysts comprise Cr(O₂CCH₃)₂(HO₂CCH₃)₂, Cr₂[CH₃(C₅H₃N)O]₄, (CrCl₂)₂.2EDA, (CrBr₂)₂EDA₂, [Cr(OH)₂]₂EDA₂ and Cr₂(O₂CCH₃)₄(H₂O)₂ a reactant displaces waters of hydration. Manganese catalysts comprise [Mn(diethyldithiocarbamate)]ₙ, (MnCl₂)₂EDA₂, K₂[Mn₂Cl₆(H₂O)₄] and Mn₂(C₅H₈O₂)₄(H₂O)₂. Iron catalysts comprise (FeCl₂)₂EDA₂ and (FeBr₂)₂EDA₂. Cobalt catalysts comprise Co₂(C₆H₅O₂)₂(C₆H₆O₂)₂, CO₂(C₅H₈O₂)₄(H₂O)₂, Co(C₆H₅O₂)₂(C₆H₆O₂)₂, Co₂(C₆H₅O₂)₄, Ca₃[Co₂(CN)₁₀]13H₂O and [Co(CN)₂]₂K₃Cu(CN)₄. Nickel catalysts comprise Ni₂(C₆H₅N₃C₆H₅), Ni₂Br₂(C₈H₆N₂) and Ni₂S₂(C₂H₂C₆H₅). Copper catalysts comprise [CuO₂CC₆H₅]₄, [CuO₂CCH₃]₄, (CuCl)₂(EtOH)₄, (CuCN)₂(EtOH)₄ and K₂Cu₄(μ₂SC₆H₅)₆.

Second and third row transition metals are organized in groups or pairs. Zirconium, hafnium, nobelium and tantalum comprise (ZrCl₂)₂, (HM1₂)₂, (HfF₂)₂, (NbCl₂)₂, (TaCl₂)₂ and (TaF₂)₂. Molybdenum and tungsten catalysts comprise [Mo(CO)₄Cl₂]₂, [W(CO)₄Cl₂]₂, [K₄MoCl₆]₂. [Mo(CN)₂]₂K₃Cu(CN)₄, [W(CN)₂]₂K₃Cu(CN)₄, [Mo(Cl)₂]₂K₃Cu(CN)₄ and [W(Cl)₂]₂K₃Cu(CN)₄. Rhenium and technetium catalysts comprise [Re(CO)₂Cl₂(PR₃)₃]₂ and [Tc(CO)₂Cl₂(PR₃)₃]₂. Platinum, palladium, ruthenium, rhodium, osmium and iridium catalysts comprise (PtF₂)₂, (PdF₂)₂, [RuCl₂]₂EDA₄, [RhCl₂]₂EDA₄, [Ru(C₈H₆N₂)₂O₂]₂, [Rh(C₈H₆N₂)₂Cl₂]₂, Ru₂(O₂CR)₄Cl, Rh₂(O₂CR)₄Cl, [PdCl₄(PBu₃)₂]₂, [PtCl₄(PBu₃)₂]₂, [OsCl₂]₂EDA₄ and [IrCl₂]₂EDA₄. Silver and gold catalysts comprise (AgCN)₂K₃Cu(CN)₄ and (AuCN)₂K₃Cu(CN)₄.

A select number of single transition metal atom catalyst complexes containing four ligands each belong to the required point group symmetry. These catalysts comprise M(II)(C₆H₅O₂)₂(C₆H₆O₂)₂, M(II)(p-C₆H₅O₂)₂, M(II)(C₆H₆NO)₂(C₆H₇NO)₂ and M(II)(O₂CCH₃)₂(HO₂CCH₃)₂ plus possible solvation ligands where M represents titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum or gold.

### Description of Catalyst Preparation and Chemical Conversion

Catalyst preparation was conducted using nitrogen purging and/or nitrogen blanketing to minimize or eliminate air oxidation of the transition metal compounds during preparation. Transition metal catalysts, effective for ambient pressure conversion of substituted organic compounds, were produced by combining transition metal salts in their lowest standard oxidation states with other reactants. Thus, such transition metal catalysts were made by partially reacting transition metal (I or II) chlorides, bromides, sulfates or cyanides with transition metal (I or II) compounds and chelates or by forming transition metal compounds in a reduced state where mono-, di-, tri- and/or poly-metal compounds result. Some examples follow.

### Example 1

The CoSO₄ catalyst was prepared by addition of 0.100 gram (1.02 mmol) of sulfuric acid dissolved in 1 mL of water to 0.249 gram (1 mmol) of Co(C₂H₃O₂)₂.4H₂O suspended in 2 mL of water with mixing. The dissolved catalyst was dried to a solid on a hot plate and used as prepared.

### Example 2

The MnSO₄ catalyst was prepared by addition of 0.100 gram (1.02 mmol) of sulfuric acid dissolved in 1 mL of water to 0.198 gram (1 mmol) of MnCl₂.4H₂O suspended in 2 mL of water with mixing. The dissolved catalyst was dried to a solid on a hot plate and used as prepared.

### Example 3

The Cr₂(SO₄)₃ catalyst was prepared by addition of 0.150 gram (1.53 mmol) of sulfuric acid dissolved in 2 mL of water to 0.266 gram (1 mmol) of CrCl₃.6H₂O suspended in 2 mL of water with mixing. The dissolved catalyst was dried to a solid on a hot plate and used as prepared.

### Example 4

The compound (VOSO₄)₂ was prepared by dispersing 1.82 grams of vanadium pentoxide in 10 grams of pure water, dissolving 3.08 grams of ammonium acetate and 4.48 grams of concentrated hydrochloric acid therein. This mixture was gently purged with nitrogen gas to displace dissolved oxygen and 6.5 grams of zinc dust was added in portions during a 5 minute period. The red brown dispersion changed to a pale blue colored solution as the catalyst formed.

Organic chemical alkylations were conducted by refluxing liquid reactants in the presence of a a small amount of catalyst and a minor amount of an acid, alkali or neutral salt dehydrating agent as described in the following examples.

### Example A

A 250 mL three neck round bottom flask was fit with a condenser, a thermometer, a nitrogen inlet tube and heated by a thermally controlled heating mantle. It was supplied with 10 grams of lime dehydrating agent, 75 grams of ethanol and approximately 0.1 gram of Co₂(C₆H₅O₂)₄ catalyst. A slow nitrogen flow was established, the heating rate set to gentle reflux and the condenser maintained at ice temperature. After two hours of heating the reaction was terminated, the flask allowed to cool to room temperature and products transferred to a sample bottle. Composition was determined by GC analysis of the liquid resulting in formation of 72% ethyl butyl ethers and 7% ethyl hexyl ether products leaving 21% of un-reacted ethanol.

### Example B

A 250 mL three neck round bottom flask was fit with a condenser, a thermometer and a nitrogen inlet tube and heated by a thermally controlled heating mantle. It was supplied with 10 grams of lime dehydrating agent, 95 grams of ethanol and approximately 0.1 gram of V₂(O₂CCH₃)₄ catalyst. A slow nitrogen flow was established, the heating rate set to gentle reflux and the condenser maintained at ice temperature. After two hours of heating the reaction was terminated, the flask allowed to cool to room temperature and products transferred to a sample bottle. Composition was determined by GC analysis of the liquid resulting in formation of 78% ethyl butyl ethers (increased n- to i-ratio), 5% ethyl hexyl ether, 1% other products and returning 16% ethanol.

### Example C

A 250 mL three neck round bottom flask was fit with a condenser, a thermometer and a nitrogen inlet tube and heated by a thermally controlled heating mantle. It was supplied with 11 grams of lime dehydrating agent, 92 grams of n-propanol and approximately 0.1 gram of Cr₂(O₂CCH₃)₄ catalyst. A slow nitrogen flow was established, the heating rate set to gentle reflux and the condenser maintained at ice temperature. After three and one quarter hours of heating the reaction was terminated, the reactor allowed to cool to room temperature and products transferred to a sample bottle. Composition was determined by GC analysis of the liquid resulting in 5% hexanol, 84% propyl hexyl ethers, 9% dihexyl ethers and 2% non-identified products.

### Example D

A 250 mL three neck round bottom flask was fit with a thermocouple, a vapor vent tube and a nitrogen inlet tube and was heated by a thermally controlled heating mantle. It was supplied with 120 mL of propylene glycol, approximately 11 grams of lime dehydrating agent and 0.07 gram of Co(O₂CCH₃)₂ hydrate catalyst. A slow nitrogen flow was established, the heating rate set to hold the reactant at 180° C. After four hours of heating the reaction was terminated, the flask allowed to cool to room temperature and products transferred to a sample bottle. Composition was determined by FTIR analysis of the liquid resulting in glycol ethers and unsaturated alcohol products.

### Example E

A 250 mL three neck round bottom flask was fit with a thermocouple, a vapor vent tube and a nitrogen inlet tube and was heated by a thermally controlled heating mantle. It was supplied with 36.5 grams of n-butylamine dissolved in 23.0 grams of ethanol, 0.7 gram of Co(O₂CCH₃)₂ hydrate catalyst and 36 grams of a calcium sulfate dehydrating agent. A slow nitrogen flow was established, the heating rate set to hold the reactant at 60° C. After five hours of heating the reaction was terminated, the flask allowed to cool to room temperature and products transferred to a sample bottle. Composition was determined by evaporative reduction and FTIR analysis of the liquid resulting in approximately 50% ethyl butyl amines, dibutyl amines, butyl hexyl amines and related amine products.

### Example F

A 125 mL conical flask was fit with a nitrogen inlet tube and was heated by a thermally controlled hot plate. It was supplied with 12.22 grams of 2,6-dimethylphenol dissolved in 4.61 grams of ethanol, 0.24 gram of Co(O₂CCH₃)₂ catalyst and 3.6 grams of a calcium sulfate dehydrating agent. A slow nitrogen flow was established, the heating rate set to hold the reactant at 60° C. Most of the reactants were lost by vaporization during the heating period. After five hours of heating the reaction was terminated, the flask allowed to cool to room temperature and liquid products transferred to a sample bottle. The products were isolated by evaporation resulting in a brown viscous liquid. Composition was determined by FTIR analysis resulting in approximately ten percent of ethylphenyl ether.

### Example G

A 125 mL conical flask was fit with a nitrogen inlet tube and was heated by a thermally controlled hot plate. It was supplied with 23.0 grams of ethanol, 29.0 grams of acetone, 0.32 gram of Co(O₂CCH₃)₂ catalyst on calcium sulfate and 30 grams of a calcium sulfate dehydrating agent. A slow nitrogen flow was established, the heating rate set to hold the mixed liquid reactants at −45° C. After two hours of heating the reaction was terminated, the flask allowed to cool to room temperature and products transferred to a sample bottle. The products were isolated by evaporating off residual reactants leaving a majority of liquid products. Composition of the liquid was determined by FTIR analysis showing production of approximately half each of methyl propyl ketone and allyl ethyl ether.

### Example H

A 40 mL glass vial was fit with a thermocouple taped to the lower exterior, a 16″×⅜″ ss tube, via a rubber stopper, to act as an air cooled condenser and placed therein 10.00 grams of anhydrous ethanol, 2.945 grams sulfuric acid dehydrating agent and 0.0178 gram of cobalt acetate tetrahydrate (formed cobalt sulfate in the reaction medium). The vial was heated open to air with initial boiling at 78° C. increasing to 103° C. at 30 minutes indicating production of butyl ethyl ether plus some 20% dibutyl ether and other ether products of a quite sharp ether odor. The majority of alkyl ether exhibited FTIR absorption bands located at 1091 and 1052 cm⁻¹ (alkyl ether), 2976, 2932, 2888, 1452, 1423, 1330 and 1276 cm⁻¹ (aliphatic hydrocarbon).

### Example I

A 40 mL glass vial was fit with a thermocouple taped to the lower exterior, a 16″×⅜″ ss tube, via a rubber stopper, to act as an air cooled condenser and placed therein 13.02 grams of anhydrous n-propanol, 2.95 grams sulfuric acid dehydrating agent and 0.022 gram of manganese chloride tetrahydrate (formed manganese sulfate in the reaction medium). The vial was heated open to air with initial boiling at 98° C. increasing to 112° C. over a 30 minute period indicating production of alkyl ether products of a quite sharp ether odor. The majority of alkyl ether exhibited FTIR absorption bands located at 1057 cm⁻¹ (alkyl ether), 2966, 2942, 2883, 1462, 1384, 1345, 1232 and 754 cm⁻¹ (aliphatic hydrocarbon). The latter band at 754 cm⁻¹ correlates with the —(CH₂)₄— motion indicating the presence of a butyl or hexyl alkyl group.

### Example J

A 40 mL glass vial was fit with a thermocouple taped to the lower exterior, a 16″×⅜″ ss tube, via a rubber stopper, to act as an air cooled condenser and placed therein 16.04 grams of n-butanol, 3.41 grams anhydrous sodium sulfate dehydrating agent and 0.017 gram of vanadyl sulfate (VOSO₄)₂. The vial was heated open to air with initial boiling at 118° C. increasing to 127° C. over a 3.5 hour period indicating production of alkyl ether products of a sharp ether odor. The majority of alkyl ether exhibited FTIR absorption bands located at 1076, 1052 cm⁻¹ (alkyl ether), 2961, 2937, 2878, 1467 and 740 cm⁻¹ (aliphatic hydrocarbon). The latter band at 740 cm⁻¹ is an absorption band correlating with the —(CH₂)₄— motion indicating the presence of a butyl or octyl alkyl group.

### Example K

A 40 mL glass vial was fit with a thermocouple taped to the lower exterior, a 16″×⅜″ ss tube, via a rubber stopper, to act as an air cooled condenser and placed therein 13.03 grams of i-propanol, 5.11 grams 75 percent phosphoric acid dehydrating agent and 0.0129 gram of chromium trichloride. The vial was heated open to air with initial boiling at 82° C. increasing to 94° C. over a 1 hour period indicating products of a quite sharp ether odor. The majority of alkyl ether exhibited FTIR absorption bands located at 1135, 1115 cm⁻¹ (alkyl ether), 2976, 2937, 2888, 1472, 1413 and 1379 cm⁻¹ (aliphatic hydrocarbon).

### Example L

A 40 mL glass vial was fit with a thermocouple taped to the lower exterior, a 16″×⅜″ ss tube, via a rubber stopper, to act as an air cooled condenser and placed therein 10.06 grams of anhydrous ethanol, 5.80 grams sulfuric acid dehydrating agent (temperature rose from 28° C. to 45° C., no apparent gas release) and 0.0167 gram of cobalt acetate tetrahydrate. The vial was heated open to air with initial boiling at 104° C. increasing to 140° C. at 30 minutes indicating production of a majority of dibutyl ether.

### Example M

A 40 mL glass vial was fit with a thermocouple taped to the lower exterior, a 16″×⅜″ ss tube, via a rubber stopper, to act as an air cooled condenser and placed therein 2.098 grams of 4-hydroxybenzoic acid, 7.455 grams of ethanol, 0.494 gram sulfuric acid dehydrating agent and 0.0192 gram of cobalt acetate tetrahydrate. The vial was heated open to air initially boiling at 83° C. and increased in 50 minutes to 97° C. Added 0.35 gram of sodium hydroxide and isolated a crystalline product. A majority of 4-ethoxybenzoic acid was produced exhibiting a mild ether odor that exhibited FTIR absorption bands located at 1242 cm⁻¹ (aryl ether), 1140 cm⁻¹ (alkyl ether), 1677, 1374, 1320, 1291 cm⁻¹ (carboxylic acid), 3088, 3034, 1613, 1594, 1169, 1110, 1018 and 856 cm⁻¹ (aromatic hydrocarbon).

### Example N

A 40 mL glass vial was fit with a thermocouple taped to the lower exterior, a 16″×⅜″ ss tube, via a rubber stopper, to act as an air cooled condenser and placed therein 5.007 grams of butylamine, 10.51 grams of butanol, 5.339 grams sulfuric acid dehydrating agent and 0.0169 gram of cobalt acetate tetrahydrate. The vial was heated open to air increasing in temperature to 130° C. at 13 minutes and 138° C. at 54 minutes. A majority of tributylamine with some dibutyl amine was produced that exhibited FTIR absorption bands located at 1022 and 1052 cm⁻¹ (tertiary amine), 2966, 2937, 2878, 1472, 1384 and 734 cm⁻¹ (aliphatic hydrocarbon) accompanied by a minor band located at 1110 cm⁻¹ (alkyl ether).

Claim of Small Entity Status

Carter Technologies is engaged in the business of chemical consultation and R&D in the field of chemical catalysis. Carter Technologies is a small business entity in accordance with 37CFR1.27 Section 1.9(c) employing less than 500 people. M K Carter of Carter Technologies qualifies as an independent inventor and does hereby assign exclusive rights to the invention, entitled Catalytic Alkylation of Alcohols to Liquid Ethers and Alkylation of Polar Organic Compounds to Alkylated Products to Carter Technologies.

The company mailing address and contact information is listed as follows,

Carter Technologies

2300 Sutter View Lane

Lincoln, Calif. 95648

Phone: (916) 543-6143

E-mail: mkcarter@ix.netcom.com

The company is owned and operated by M K Carter, PhD residing at

2300 Sutter View Lane

Lincoln, Calif. 95648

I testify these disclosures are true as stated above,

M K Carter

Feb. 1, 2012

I stand as a true witness to the authenticity of the signature of M K Carter,

Judy Carter

Feb. 1, 2012

Assignment of Patent Ownership

M K Carter, an independent inventor, has conceived of and reduced to practice certain catalysts and a process in the field of catalysis entitled, Catalytic Alkylation of Alcohols to Liquid Ethers and Organic Compounds to Alkylated Products, application Ser. No. 13/385,192, hereinafter referred to as the invention.

Carter Technologies is engaged in the business of chemical consultation and R&D in the field of chemical catalysis.

M K Carter qualifies as an independent inventor and does hereby assign exclusive rights to the invention to Carter Technologies in accord with 37CFR3.73 (b).

The company mailing address and contact information is listed as follows,

Carter Technologies

2300 Sutter View Lane

Lincoln, Calif. 95648

Phone: (916) 543-6143

E-mail: mkcarter@ix.netcom.com

M K Carter, PhD resides at

2300 Sutter View Lane

Lincoln, Calif. 95648

I testify these disclosures are true as stated above,

M K Carter

Feb. 1, 2012

I stand as a true witness to the authenticity of the signature of M K Carter,

Judy Carter

Feb. 1, 2012

Accounting of US patent application fees for patent application entitled, Catalytic Alkylation of Alcohols to Liquid Ethers and Organic Compounds to Alkylated Products.

The following information was extracted from the UNITED STATES PATENT AND TRADEMARK OFFICE web site; FEE SCHEDULE, Effective Sep. 26, 2011 (Last Revised on Jan. 10, 2012). The filing fee (or national fee), search fee, and examination fee are due on filing.

| | | | Small Entity Price |
|---|---|---|---|
| Fee Type | Fee Code | 37CFR entry | ($) |
| Basic filing fee | 1011/2011 | 1.16(a)(1) | 190. |
| | 1090/2090 | 1.16(t) | 200. |
| Independent claims | 1201/2201 | 1.16(h) | 125./claim |
| in excess of three | | | |
| Utility Search Fee | 1111/2111 | 1.16(k) | 310. |
| Utility Examination Fee | 1311/2311 | 1.16(o) | 125. |
| Total Fees | — | — | 2,450. |

A check is enclosed to the Director of the U.S. Patent and Trademark Office in the amount of $750.00. A second check was sent in response of Incomplete Application to the Director of the U.S. Patent and Trademark Office in the amount of $1,700.00.

Submit to:

United States Patent and Trademark Office

Customer Service Window

Randolph Building

401 Dulany Street

Alexandria, Va. 22314

Submitted patent application with check number 3090 in the amount of $750.00 to

Commissioner of Patents

P.O. Box 1450

Alexandria, Va. 22313-1450

What is claimed:

1. Alkylation of ethanol by ethanol refluxing at a temperature below 200° C. forming ether compounds comprising ethyl butyl ether, dibutyl ether and ethyl hexyl ether in the presence of a catalyst and a minor amount of dehydrating agent, at ambient pressure in the absence of air and zero valent metals.

2. Alkylation of ethanol by ethanol refluxing at a temperature below 200° C. forming ether compounds comprising ethyl butyl ether, dibutyl ether and ethyl hexyl ether in the presence of a catalyst wherein catalysts, based on selected mono-metal, di-metal, tri-metal and/or poly-metal backbone or molecular string type transition metal complexes possessing a degree of symmetry being in C₄ᵥ, D_{2d} or D₄ₕ point group molecular symmetry configuration, are formed from transition metal compounds in a low oxidation state, such as 2+, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, for example vanadium(II)]₂, [chromium(II)]₂, [manganese(II)]₂ and [cobalt(II)]₂ sulfate, with a minor amount of dehydrating agent, at ambient pressure in the absence of air and zero valent metals.

3. Alkylation of propanol by propanol refluxing at a temperature below 200° C. forming ether compounds comprising propyl hexyl ether, dihexyl ether and propyl nonyl ether in the presence of a catalyst and a minor amount of dehydrating agent, at ambient pressure in the absence of air and zero valent metals.

4. Alkylation of propanol by propanol refluxing at a temperature below 200° C. forming ether compounds comprising propyl hexyl ether, dihexyl ether and propyl nonyl ether in the presence of a catalyst wherein catalysts, based on selected mono-metal, di-metal, tri-metal and/or poly-metal backbone or molecular string type transition metal complexes possessing a degree of symmetry being in C₄ᵥ, D_{2d} or D₄ₕ point group molecular symmetry configuration, are formed from transition metal compounds in a low oxidation state, such as 2+, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, for example vanadium(II)]₂, [chromium(II)]₂, [manganese(II)]₂ and [cobalt(II)]₂ sulfate, with a minor amount of dehydrating agent, at ambient pressure in the absence of air and zero valent metals.

5. Alkylation of butanol by butanol refluxing at a temperature below 200° C. forming ether compounds comprising butyl octyl ether, dioctyl ether and butyl dodecyl ether in the presence of a catalyst and a minor amount of dehydrating agent, at ambient pressure in the absence of air and zero valent metals.

6. Ambient pressure alkylation of butanol by butyl groups produced from butanol for formation of ether compounds comprising butyl octyl ether, dioctyl ether and butyl dodecyl ether in the presence of a catalyst wherein catalysts, based on selected mono-metal, di-metal, tri-metal and/or poly-metal backbone or molecular string type transition metal complexes possessing a degree of symmetry being in C₄ᵥ, D_{2d} or D₄ₕ point group molecular symmetry configuration, are formed from transition metal compounds in a low oxidation state, such as 2+, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, for example vanadium(II)]₂, [chromium(II)]₂, [manganese(II)]₂ and [cobalt(II)]₂ sulfate, with a minor amount of dehydrating agent, at ambient pressure in the absence of air and zero valent metals.

7. Alkylation of butyl amine by ethanol refluxing at a temperature below 200° C. forming compounds comprising ethyl butyl amine, butyl diethyl amine, dibutyl amine and ethyl hexyl amine in the presence of a catalyst and a minor amount of dehydrating agent, at ambient pressure in the absence of air and zero valent metals.

8. Alkylation of butyl amine by ethanol refluxing at a temperature below 200° C. forming compounds comprising ethyl butyl amine, butyl diethyl amine, dibutyl amine and ethyl hexyl amine in the presence of a catalyst wherein catalysts, based on selected mono-metal, di-metal, tri-metal and/or poly-metal backbone or molecular string type transition metal complexes possessing a degree of symmetry being in C₄ᵥ, D_{2d} or D₄ₕ point group molecular symmetry configuration, are formed from transition metal compounds in a low oxidation state, such as 2+, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, for example vanadium(II)ₕ, [chromium(II)]₂, [manganese(II)]₂ and [cobalt(II)]₂ sulfate, with a minor amount of dehydrating agent, at ambient pressure in the absence of air and zero valent metals.

9. Alkylation of 2,6-dimethylphenol by ethanol refluxing at a temperature below 200° C. forming ether compounds of 2,6-dimethyl ethyl phenyl ether in the presence of a catalyst and a minor amount of dehydrating agent, at ambient pressure in the absence of air and zero valent metals.

10. Alkylation of 2,6-dimethylphenol by ethanol refluxing at a temperature below 200° C. forming ether compounds of 2,6-dimethyl ethyl phenyl ether in the presence of a catalyst wherein catalysts, based on selected mono-metal, di-metal, tri-metal and/or poly-metal backbone or molecular string type transition metal complexes possessing a degree of symmetry being in C₄ᵥ, D_{2d} or D₄ₕ point group molecular symmetry configuration, are formed from transition metal compounds in a low oxidation state, such as 2+, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, for example vanadium(II)]₂, [chromium(II)]₂, [manganese(II)]₂ and [cobalt(II)]₂ sulfate, with a minor amount of dehydrating agent, at ambient pressure in the absence of air and zero valent metals.

11. Alkylation of acetone by ethanol refluxing at a temperature below 200° C. forming compounds comprising 2-pentanone, 2-heptanone and 4-heptanone in the presence of a catalyst and a minor amount of dehydrating agent, at ambient pressure in the absence of air and zero valent metals.

12. Alkylation of acetone by ethanol refluxing at a temperature below 200° C. forming compounds comprising 2-pentanone, 2-heptanone and 4-heptanone in the presence of a catalyst wherein catalysts, based on selected mono-metal, di-metal, tri-metal and/or poly-metal backbone or molecular string type transition metal complexes possessing a degree of symmetry being in C₄ᵥ, D_{2d} or D₄ₕ point group molecular symmetry configuration, are formed from transition metal compounds in a low oxidation state, such as 2+, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, for example vanadium(II)]₂, [chromium(II)]₂, [manganese(II)]₂ and [cobalt(II)]₂ sulfate, with a minor amount of dehydrating agent, at ambient pressure in the absence of air and zero valent metals.

13. Alkylation of 4-hydroxybenzoic acid by ethanol for formation of compounds comprising 4-ethoxybenzoic acid at a reflux temperature below 200° C. in the presence of a catalyst and a minor amount of dehydrating agent, at ambient pressure in the absence of air and zero valent metals.

14. Alkylation of 4-hydroxybenzoic acid by ethanol for formation of compounds comprising 4-ethoxybenzoic acid at a reflux temperature below 200° C. in the presence of a catalyst wherein catalysts, based on selected mono-metal, di-metal, tri-metal and/or poly-metal backbone or molecular string type transition metal complexes possessing a degree of symmetry being in C₄ᵥ, D_{2d} or D₄ₕ point group molecular symmetry configuration, formed from transition metal compounds, in a low oxidation state, such as 2+, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, for example vanadium(II)]₂, [chromium(II)]₂, [manganese(II)]₂ and [cobalt(II)]₂ sulfate, with a minor amount of dehydrating agent, at ambient pressure in the absence of air and zero valent metals.

15. Alkylation of organic compounds, comprising alcohols, alkanes, glycols, ethers, aldehydes, ketones, carboxylic acids, esters, amines, thiols or phosphines, by an alcohol or glycol refluxing at a temperature below 200° C. forming ether and higher molecular weight compounds in the presence of a catalyst and a minor amount of dehydrating agent, at ambient pressure in the absence of air and zero valent metals.

16. Alkylation of organic compounds, comprising alcohols, alkanes, glycols, ethers, aldehydes, ketones, carboxylic acids, esters, amines, thiols or phosphines, by an alcohol or glycol refluxing at a temperature below 200° C. in the presence of a catalyst wherein catalysts, based on selected mono-metal, di-metal, tri-metal and/or poly-metal backbone or molecular string type transition metal complexes possessing a degree of symmetry being in C₄ᵥ, D_{2d} or D₄ₕ point group molecular symmetry configuration, are formed from transition metal compounds in a low oxidation state, such as 2+, comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, for example vanadium(II)]₂, [chromium(II)]₂, [manganese(II)]₂ and [cobalt(II)]₂ sulfate, with a minor amount of dehydrating agent, at ambient pressure in the absence of air and zero valent metals.
